(19) United States
(12) Patent Application Publication
(10) Pub. No.: US 2006/0149055 A1
(43) Pub. Date: Jul. 6, 2006

(54) Process for the manufacture of cefpodoxime proxetil

(76) Inventors: Milind Moreshwar Gharpure, Pune (IN); Sanjay Shankar Deshmukh, Pune (IN); Rajendra Dagesing Mahale, Pune (IN)

Correspondence Address: LADAS & PARRY, 26 WEST 61ST STREET, NEW YORK, NY, 10023, US

(21) Appl. No.: 10/541,473

(22) PCT Filed: Jan. 6, 2003
(86) PCT No.: PCT/IN03/00003

§ 371 Date: Jan. 19, 2006

(51) Int. Cl. C07D 501/14

(52) U.S. Cl. 540/222

(57) ABSTRACT

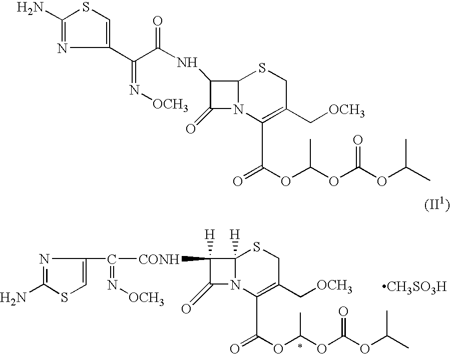

A process for obtaining cefpodoxime proxetil of formula (I), of high purity conforming to pharmacopoeial specifications is disclosed. The process comprises addition of a solution of methanesulfonic acid in water to a solution of impure cefpodoxime proxetil of formula (I) in an organic solvent to form the corresponding cefpodoxime proxetil methanesulfonate of formula (II¹), followed by addition of a co-solvent and separation of the aqueous phase containing cefpodoxime proxetil methanesulfonate of formula (II¹) having a diastereomeric ratio of (R/R+S) between 0.5 to 0.6 and subsequent neutralization of the methaneulfonate salt (II¹) with a base to give cefpodoxime proxetil (I) free of impurities and having a diastereomeric ratio of (R/R+S) between 0.5 to 0.6, or, addition of impure cefpodoxime proxetil of formula (I) to a solution of methanesulfonic acid in water to form the corresponding solution of cefpodoxime proxetil methanesulfonate of formula (II¹) in water, followed by sequential addition of a first organic solvent and a co-solvent and separation of the aqueous phase containing cefpodoxime proxetil methanesulfonate of formula (II¹) having a diastereomeric ratio of (R/R+S) between 0.5 to 0.6 and subsequent neutralization of the methaneulfonate salt (II¹) with a base to give cefpodoxime proxetil (I) free of impurities and having a diastereomeric ratio of (R/R+S) between 0.5 to 0.6.

### FIELD OF THE INVENTION

The present invention provides a simple method for obtaining cefpodoxime proxetil of high purity conforming to pharmacoepeial specifications.

### BACKGROUND OF THE INVENTION

Cefpodoxime Proxetil of formula (I), chemically known as 1-isopropoxycarbonyloxyethyl(6R,7R)-7-[2-(2-aminothiazol-4-yl)-2(Z)-(methoxyimino)acetamido]-3-(methoxymethyl)-3-cephem-4-carboxylate belongs to the third generation of cephalosporin antibiotics, which is administered orally.

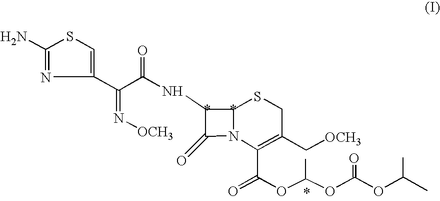

Cefpodoxime proxetil of formula (I) has two asymmetric centers at position 6 and 7 of the cephem nucleus and another one at the α-carbon of 1-isopropoxycarbonyloxyethyl group attached to the 4-carboxyl group as shown in the above structure. The asymmetric center of the α-carbon of 1-isopropoxycarbonyloxyethyl group attached to the 4-carboxyl group exists as a pair of diastereoisomers, notated as the R and S isomers. Pharmacopoeial Forum Vol: 28 (1), pp 44-52, (2002) mentions that the diastereomeric ratio (R/R+S) of cefpodoxime proxetil should be between 0.5 to 0.6.

U.S. Pat. No. 4,486,425 (Nakao, et al) discloses several methods for preparation of cefpodoxime proxetil of formula (I). However, all the methods are associated with formation of varying amounts of impurities. The associated impurities have been removed by taking recourse to chromatographic separation techniques, which albeit provides the compound of formula (I) of desired quality. However, such method is cumbersome and not practical on industrial scale.

The impurities which are normally associated with manufacturing processes for cefpodoxime proxetil, to name a few, are the Δ² isomer and the E-isomer of cefpodoxime proxetil which have the chemical structures shown below,

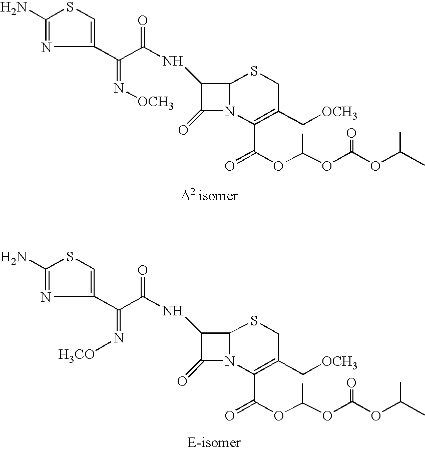

Journal of Antibiotics (1987, Vol 40, 370, Fujimoto. K et. al) describes another method for preparation of cefpodoxime proxetil which consists of the conversion of 7-[2-(2-chloroacetyl amino thiazolyl)-2-(2-methoxyimino acetamido]-3-acetoxy-methyl-3-cephem-4-carboxylic acid (V) into the corresponding 3-methoxymethyl derivative, by reaction of methanol and CaCl₂.2H₂O, which on deprotection of the amino protecting group at the 2-aminothiazolyl ring gives cefpodoxime acid (VI).

Esterification of the carboxylic acid with 1-iodoethylisopropyl carbonate gives the compound of formula (I).

However, this method also takes recourse to chromatographic methods for obtaining the product in pure form. Moreover, this method also involves additional steps of protection and deprotection of the amino group resulting in overall lower efficiency.

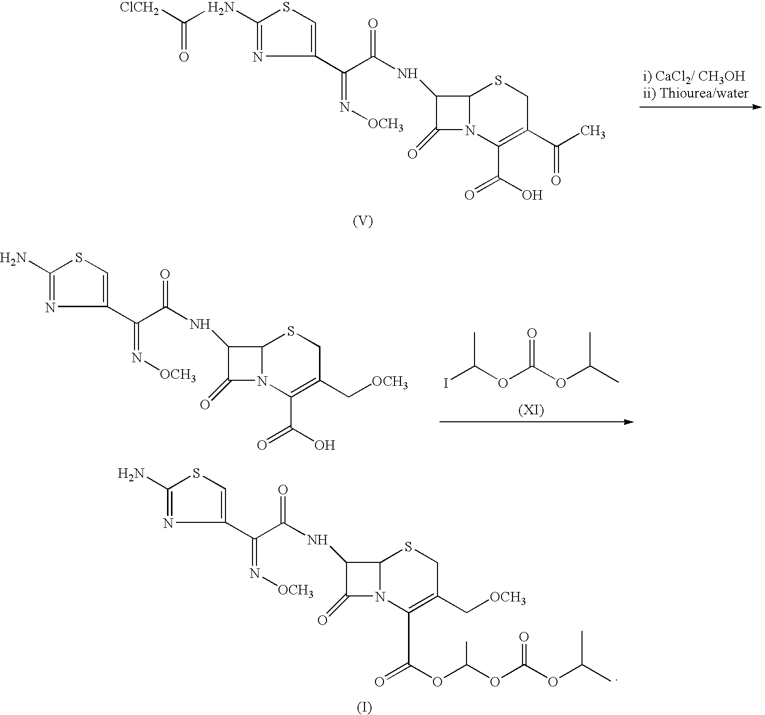

PCT Application No. WO 99/35149 A1 (Greil et. al) describes yet another method for preparation of cefpodoxime proxetil of formula (I), wherein, Z-2-(methoxyimino)-2-(2-formylaminothiazol-4-yl)acetic acid (VII) activated as the acid chloride hydrochloride on reaction with a compound of formula (VIII), gives N-formylcefpodoxime proxetil of formula (IX).

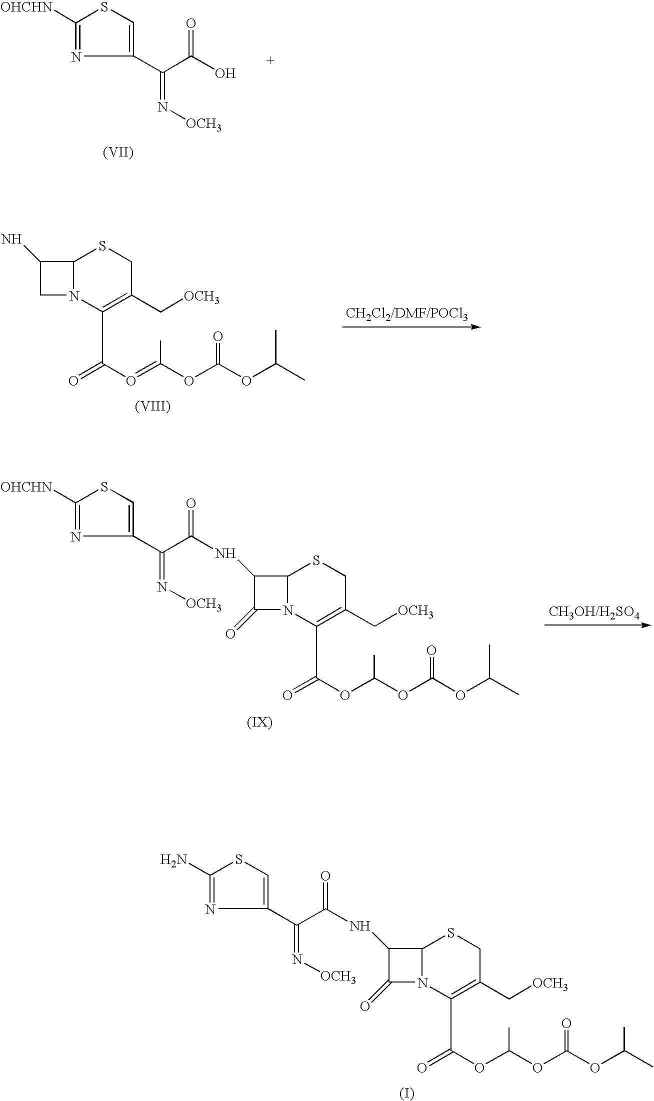

In this method, the intermediate compound of formula (IX) is purified by treating it with water/alcohol mixture containing an additive, such as an organic amide, urea, an imidazolidinone or a pyrimidinone. The purified compound (IX) thus obtained on treatment with acid gives cefpodoxime proxetil of formula (I).

Even though, this method does not involve purification through column chromatography in the final step, however additional steps of protection of the amino group as the formyl derivative and its subsequent deprotection are required.

US Patent Application No. 2002/0065262 A1 (Greil. J, et al) describes a method for preparation of cefpodoxime proxetil having the right diastereomeric ratio and chemical purity via the intermediacy of a crystalline form of 7-[2-(2-formylaminothiazol-4-yl)-2-(Z)-(methoxyimino) acetamido]-3-methoxymethyl-3-cephem-4-carboxylic acid-1-(isopropoxycarbonyloxy)ethyl ester of formula (IX).

The preparation of the crystalline form of compound (IX) comprises reaction of 7-amino-3-methoxymethyl-3-cephem-4-carboxylic acid (X) with 1-iodoethyl isopropyl carbonate (XI) to give the compound (VIII), which on condensation with Z-(2-formylaminothiazo-4-yl)-methoxyimino acetic acid activated as an ester or a halogenide gives N-formyl cefpodoxime proxetil, which is then crystallized from a mixture of a nitrile or a ketonic solvent with water to get crystalline N-formyl cefpodoxime of formula (IX) having a diastereomeric ratio between 0.5 to 0.6.

The compound (IX) is then treated with H₂SO₄/methanol to get cefpodoxime proxetil having isomeric ratio between 0.5 to 0.6.

This method also requires a protection and a deprotection step in addition to a step of crystallisation to give cefpodoxime proxetil, which not only is lengthy but decreases the overall yield.

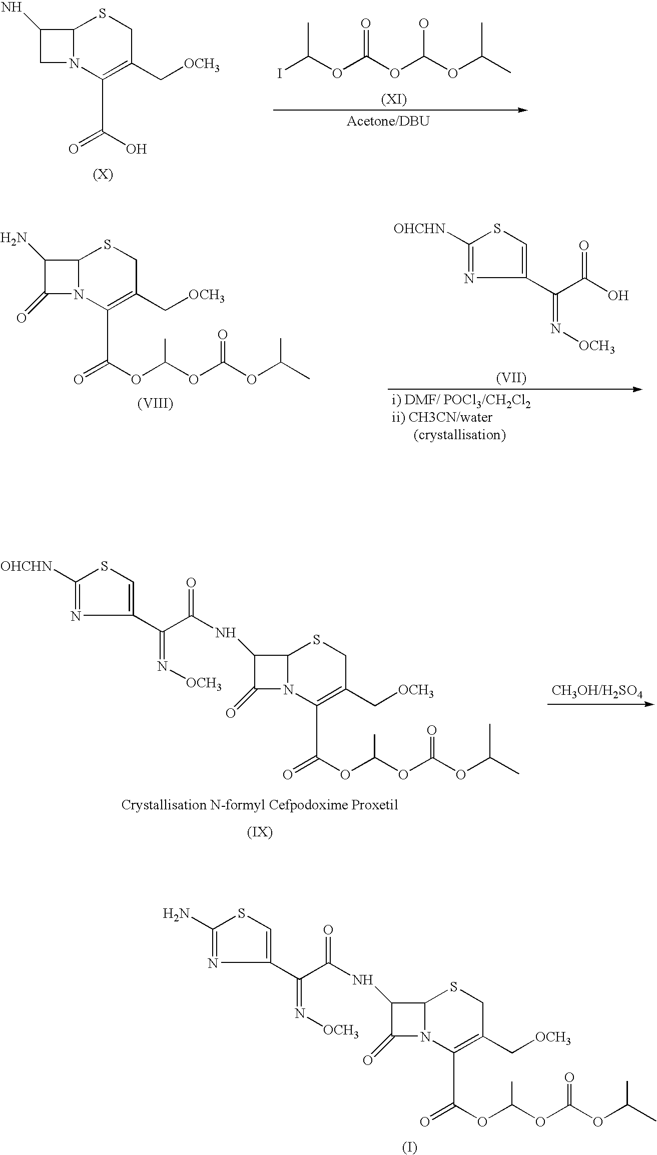

PCT Application No. WO 02/068429 A1 (Yatendra Kumar, et. al.) discloses a method for purification and isolation of cefpodoxime proxetil, obtained by reacting cefpodoxime acid of formula (VI), with 1-iodoethyl isopropylcarbonate of formula (XI) in the presence of a base.

The purification of impure cefpodoxime proxetil thus obtained is carried out in two stages which comprises of;
i) dissolving impure cefpodoxime proxetil or adding a solution containing cefpodoxime proxetil into a polar organic solvent or mixture(s) thereof optionally reducing the solvent by concentration, and adding into a non-polar organic solvent or mixture(s) thereof to precipitate the solid; and
ii) dissolving the solid obtained from the above step into a water miscible polar organic solvent, optionally reducing the solvent by concentration, and adding into water to obtain the pure cefpodoxime proxetil.

However, this method involves a two-step isolation and purification, which moreover involves use of a combination of solvents for crystallization, rendering the method tedious and not commercially attractive.

Moreover, in most of the methods mentioned hereinbefore, the ratio of the diastereomeric isomers, (R/R+S) of cefpodoxime proxetil obtained is not found to be the prescribed range between 0.5 to 0.6 and additional steps are involved to adjust the ratio to the desired levels.

To summarize, cefpodoxime proxetil prepared by various methods as mentioned herein earlier, is invariably contaminated with varying amounts of impurities and varying proportions of the diastereomeric ratio of the R and S isomers associated with the respective method of preparation.

The methods employed for obtaining cefpodoxime proxetil of desired quality and specification utilize,
i) separation techniques like chromatography,
ii) protection and deprotection of reactive functional groups,
iii) multi-step crystallization,
iv) use of combination of solvents for crystallization, and
v) adjustment of the diastereomeric R and S ratio,
   which render such methods less suitable for industrial application.

Therefore, a need exists for a simple, cost-effective method for obtaining cefpodoxime proxetil of high purity, which overcomes the shortcomings of the prior art methods.

Such a need has been met by the present invention, which is described in detail herein below.

### OBJECTS OF THE INVENTION

Accordingly, an object of the present invention is to provide a simple method for preparation of cefpodoxime proxetil of high purity.

Another object of the present invention is to provide a simple, cost-effective method for preparation of cefpodoxime proxetil of high purity.

Yet another aspect of the present invention is to provide a simple, selective and cost-effective method for preparation of cefpodoxime proxetil of high purity and conforming to pharmacoepeial specifications

### SUMMARY OF THE INVENTION

The invention consists of reaction of impure cefpodoxime proxetil with a strong acid to form a salt of cefpodoxime proxetil of formula (II), 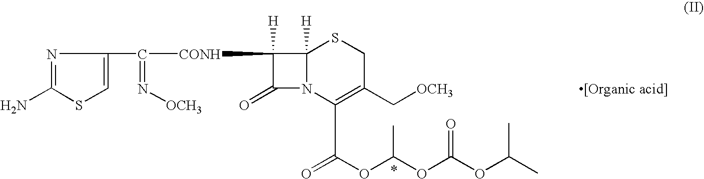
comprising addition of a solution of the organic acid in water to a solution of cefpodoxime proxetil in a water-immiscible organic solvent, wherein the salt of formula (II) thus formed preferentially gets solubilised in water and partly in the organic solvent, leaving the impurities such as the Δ²-isomer and the E-isomer and others formed during the synthesis of the antibiotic compound (I) selectively in the organic solvent.

The salt of formula (II) partly partitioned in the organic phase is brought into the aqueous phase by addition of a co-solvent, followed by neutralization of the aqueous solution containing the salt (II) with a base by conventional methods to give cefpodoxime proxetil (I) of high purity conforming to pharmacoepeial specifications.

The product thus obtained can optionally be dissolved in a water-miscible organic solvent, and the solution either charcoalised or non-charcoalised is filtered through a filtering aid to remove carbon and suspended particles, followed by addition of water to precipitate out the cefpodoxime proxetil, which can be isolated by filtration.

Surprisingly, strong acids like hydrochloric, hydrobromic, sulfuric, p-toluene sulfonic, benzenesulfonic, trifluoroacetic, etc do not make the corresponding salts of cefpodoxime proxetil with these acids preferentially soluble in water, but only methanesulfonic acid renders the salt obtained from thereof i.e. cefpodoxime proxetil methanesulfonate of formula (II¹) highly soluble in water with concomitant partitioning of all impurities in the organic phase.

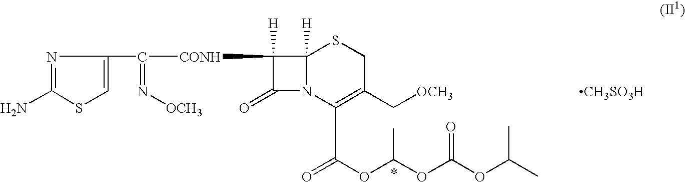

Thus, the above and other objects of the present invention are achieved by the process of the present invention comprising the steps of:
addition of a solution of methanesulfonic acid in water to a solution of impure cefpodoxime proxetil of formula (I) 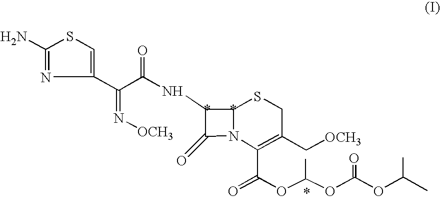
in an organic solvent to form the corresponding cefpodoxime proxetil methanesulfonate of formula (II¹) 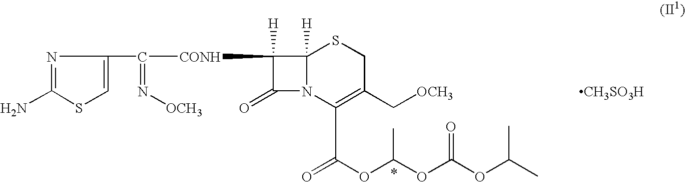
followed by addition of a co-solvent and separation of the aqueous phase containing cefpodoxime proxetil methanesulfonate of formula (II¹) having a diastereomeric ratio of (R/R+S) between 0.5 to 0.6 and subsequent neutralization of the methaneulfonate salt (II¹) with a base to give cefpodoxime proxetil (I) free of impurities and having a diastereomeric ratio of (R/R+S) between 0.5 to 0.6,
or
   addition of impure cefpodoxime proxetil of formula (I) 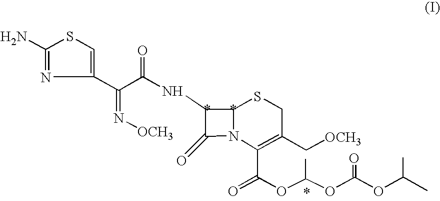
   to a solution of methanesulfonic aicd in water to form the corresponding solution of cefpodoxime proxetil methanesulfonate of formula (II¹) in water, 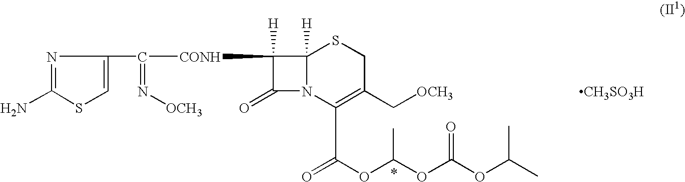
   followed by sequential addition of an organic solvent and a co-solvent and separation of the aqueous phase containing cefpodoxime proxetil methanesulfonate of formula (II¹) having a diastereomeric ratio of (R/R+S) between 0.5 to 0.6 and subsequent neutralization of the methaneulfonate salt (II¹) with a base to give cefpodoxime proxetil (I) free of impurities and having a diastereomeric ratio of (R/R+S) between 0.5 to 0.6,

Preferably, in an optional embodiment, the pure cefpodoxime proxetil obtained above is subjected to a further purification step. This is achieved by optionally dissolving the pure cefpodoxime proxetil (I) as obtained above in a water-miscible organic solvent, followed by optional treatment of the solution with activated charcoal followed by filtration through a filter aid to remove charcoal and suspended particles and addition of water to the filtrate to precipitate out cefpodoxime proxetil (I) free of impurities and having a diastereomeric ratio of (R/R+S) between 0.5 to 0.6, which can be isolated by filtration.

### DETAILED DESCRIPTION OF THE INVENTION

The method for preparation of cefpodoxime proxetil of formula (I) in accordance with the present invention comprises of the following steps, which are summarized in Scheme-I.

Synthesis of Impure Cefpodoxime Proxetil of Formula (I)

The first step consists of reacting cefpodoxime acid of formula (VI) with 1-iodoethyl isopropylcarbonate of formula (I), to give cefpodoxime proxetil of formula (I).

Typically, cefpodoxime acid of formula (VI) (prepared by any of the general methods reported in U.S. Pat. No. 4,486,425) is dissolved in a aprotic solvent such as N,N-dimethyl acetamide, N,N-dimethyl formamide, dimethyl sulphoxide etc., preferably N,N-dimethyl acetamide which is in a ratio of 5-10 times volume per gram of cefpodoxime acid taken.

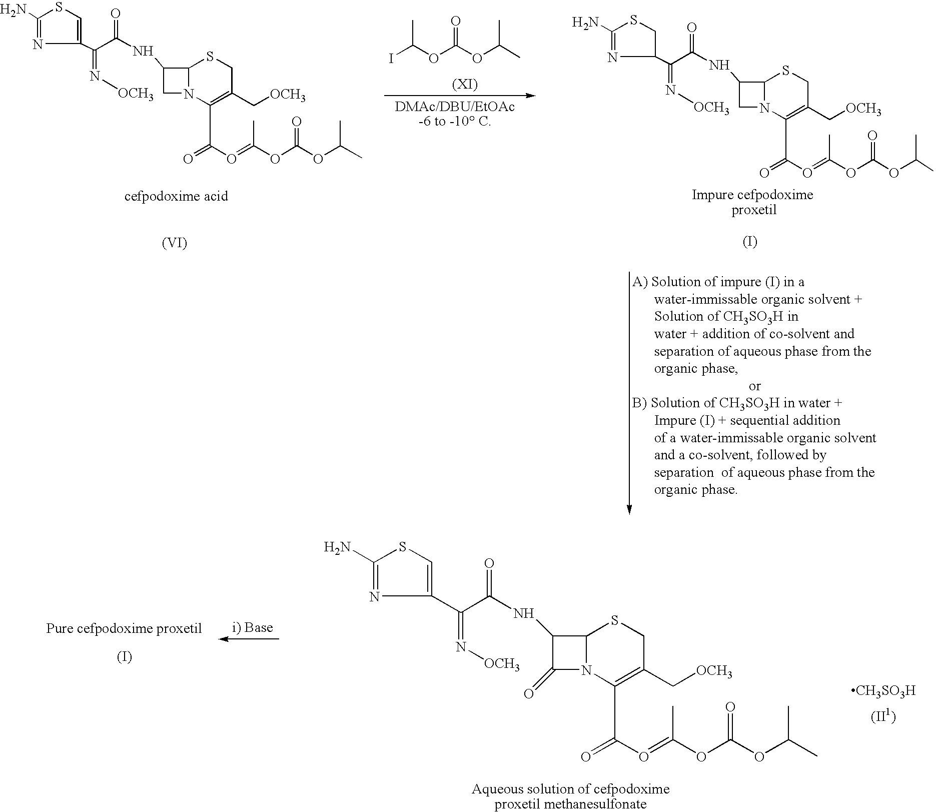

The acid (VI) is converted to its salt using a base selected from a group of inorganic bases like potassium carbonate, sodium carbonate, sodium bicarbonate etc., preferably sodium carbonate, or from a group of organic bases like dicyclohexyl amine, 1,8-diazabicyclo[5,4,0]undec-7-ene (DBU), etc., preferably DBU, or a mixture thereof.

The molar ratio of the base used is ideally in the range of 0.95 to 1.02 mole equivalent, but preferably between 0.96 to 0.98 mole equivalent with respect to the acid (VI) used.

The salt formation can be carried out at temperatures between −20° to 0° C., but preferably between −5° C. to −10° C.

1-Iodoethylisopropyl carbonate of formula (XI) is then added to the solution of salt of the acid (VI) thus obtained in an aprotic solvent at a temperature ranging between −20° C. to 0° C., but preferably between −5° C. to −10° C. The molar ratio of 1-Iodoethylisopropyl carbonate used is in the range of 1.0 to 1.2 moles.

The duration of addition of 1-iodoethylisopropyl carbonate of formula (XI) can be between 5 to 30 minutes, but preferably between 5 to 15 minutes.

The reaction can be carried out at a temperature ranging between −20 to 0° C., but preferably between −5 to −10° C. The reaction is normally over in 10 to 60 minutes.

After completion of the reaction, the reaction mixture is quenched with a dilute inorganic acid like sulphuric acid, hydrochloric acid etc., but preferably dilute hydrochloric acid.

The reaction mixture can then be extracted with a water-immiscible organic solvent, selected from alkyl acetates and ketonic solvents. Suitable alkyl acetates include methyl acetate, ethyl acetate, and butyl acetate and the ketonic solvents include methyl ethyl ketone (MEK) and methyl iso-butyl ketone (MIBK). Of these, ethyl acetate and MIBK are preferred.

The organic layer containing cefpodoxime proxetil thus formed is washed with a dilute aqueous alkali metal carbonate solution, like sodium carbonate, sodium bicarbonate, potassium carbonate etc., but preferably with a dilute aqueous solution of sodium bicarbonate. The temperature during the alkali wash should be in the range of −5 to +5° C. but preferably between 0 to 5° C.

The organic layer is subsequently washed with 5% sodium thiosulphate solution followed by water.

The organic layer is optionally treated with carbon and filtered. The filtrate can be evaporated to give the impure cefpodoxime proxetil (I), which can be used for further purification steps detailed hereinbelow.

Alternatively, the solution of the impure cefpodoxime proxetil in the organic solvent can be partially evaporated and the concentrated solution can as such be used for further purification steps detailed hereinbelow.

Alternatively, the reaction mixture after completion of reaction can be dumped into water and the solid precipitated can be filtered off to give impure cefpodoxime proxetil. This solid material as such can be used for further purification steps detailed hereinbelow.

In a specific embodiment of the invention, a clear solution of cefpodoxime acid (VI) in dimethyl acetamide is cooled to −5° C. and 1,8-diazabicyclo[5,4,0]undec-7-ene (DBU) in stoichiometric amounts is added in 10 minutes at the same temperature. The reaction mixture is agitated for 5-10 minutes after the addition, for complete salt formation after which 1-iodoethylisopropyl carbonate (XI) is added at 6 to −10° C. in 10 minutes.

The reaction mixture is agitated for 20 to 30 minutes at 6 to −8° C., quenched with 10% hydrochloric acid and the aqueous layer extracted with ethyl acetate.

The reaction mixture is agitated for 20 to 30 minutes at 6 to −8° C. for the reaction to be completed. The reaction mixture is worked up by quenching with dilute hydrochloric acid and extracting the aqueous layer successively with ethyl acetate.

The organic layer is agitated with 2% sodium carbonate solution, at 0-5° C. for 30 minutes and then further washed with 5% sodium thiosulphate solution.

The organic layer is optionally treated with activated carbon and filtered. The filtrate can be evaporated to give the impure cefpodoxime proxetil (I), which can be used for further purification steps detailed hereinbelow.

Alternatively, the solution of the impure cefpodoxime proxetil in the organic solvent can be partially evaporated and the concentrated solution can as such be used for further purification steps detailed hereinbelow.

Alternatively, the reaction mixture after completion of reaction can be dumped into water and the solid precipitated can be filtered off to give impure cefpodoxime proxetil. This solid material as such can be used for further purification steps detailed hereinbelow.

Purification of Impure Cefpodoxime Proxetil

The impure cefpodoxime proxetil (I) obtained in the previous step is reacted with a strong organic acid to form the salt of cefpodoxime proxetil of formula (II) with the organic acid employed.

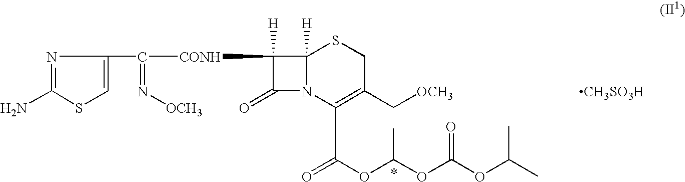

The formation of the salt of formula (II) comprises reaction of a solution of the impure cefpodoxime proxetil (isolated in the previous step by evaporation of the solvent from which it was extracted from the reaction mixture) in an organic solvent with a solution of the organic acid in water to form the salt thereof.

Alternatively, the salt can also be conveniently prepared by treating the solution of impure cefpodoxime proxetil (obtained by partial evaporation of the solvent from which the impure compound was extracted from the reaction mixture in the previous step) with a solution of the organic acid in water to form the salt thereof.

Alternatively, the solid impure cefpodoxime proxetil obtained in the previous step by dumping the reaction mixture into water, after drying or preferably without drying can be added to a solution of the strong acid in water to form the salt thereof. In this case an organic solvent is further added to the aqueous solution for effective partitioning of the salt and the impurities in the two phases.

The salt thus formed is selectively portioned in one of the two phases. Surprisingly, strong acids like hydrochloric, hydrobromic, sulfuric, p-toluene sulfonic, benzenesulfonic, trifluoroacetic, etc do not make the corresponding salts of cefpodoxime proxetil with these acids preferentially soluble in water, but only methanesulfonic acid renders the salt obtained from thereof i.e. cefpodoxime proxetil methanesulfonate of formula (II¹) highly soluble in water with concomitant partitioning of all impurities in the organic phase.

Thus in a specific embodiment of this invention the formation of the methanesulfonate salt of formula (II¹) comprises: 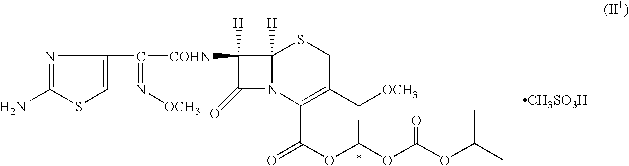
reaction of a solution of the impure cefpodoxime proxetil (isolated in the previous step by evaporation of the solvent from which it was extracted from the reaction mixture) in an organic solvent with a solution of methanesulfonic acid in water to form the salt (II¹) thereof, or
reaction of a solution of impure cefpodoxime proxetil (obtained by partial evaporation of the solvent from which the impure compound was extracted from the reaction mixture in the previous step) with a solution of methanesulfonic acid in water to form the salt (II¹) thereof or alternatively,
addition of the solid impure cefpodoxime proxetil, obtained in the previous step by dumping the reaction mixture into water, after drying or preferably without drying can be added to a solution of methanesulfonic acid in water to form the salt (II¹) thereof. In this case an organic solvent is further added to the aqueous solution for effective partitioning of the salt and the impurities in the two phases.

The salt forming reaction can be carried out in the temperature range of 10 to 35° C., but preferably at 25 to 30° C.

The molar ratio of the methanesulfonic acid used for forming the salt (II¹) is in the range of 1.0 to 2.0 mole equivalent of cefpodoxime proxetil, but preferably between 1.5 to 2.0 mole equivalent.

The methanesulfonate salt (II¹) thus obtained is preferentially solubilised in water and partially in the organic solvent, leaving all the impurities in the organic phase. The salt that is partially portioned in the organic phase is brought back into the aqueous phase by addition of a co-solvent. The total salt that is brought back to the aqueous phase and which remains soluble in water has a diastereomeric ratio of (R/R+S) between 0.5 to 0.6.

The organic solvents that can be used for the salt forming reaction and selective partitioning of the impurities are water-immiscible and are the ones that are used for extraction of impure cefpodoxime proxetil from the reaction mixture. These solvents are selected from alkyl acetates and ketonic solvents. Suitable alkyl acetates include methyl acetate, ethyl acetate, and butyl acetate and the ketonic solvents include methyl ethyl ketone (MEK) and methyl iso-butyl ketone (MIBK). Of these, ethyl acetate and MIBK are preferred.

The organic solvent is used is between 2 to 7 times of volume to the weight of impure cefpodoxime proxetil taken, but the preferred volume is in the range of 2 to 4 times the weight of impure cefpodoxime proxetil.

The amount of water used for forming an aqueous solution of methanesulphonic acid is in the range of 40-60 times volume by wt of methanesulfonic acid used.

The co-solvents that can be used for the salt forming reaction and selective partitioning of the impurities are water-immiscible and are ideally non-polar solvents selected from aliphatic hydrocarbons, both straight and cyclic; aromatic hydrocarbons and ethers include diethyl ether, diisopropyl ether etc.

Among all these solvents, aliphatic hydrocarbons and among aliphatic hydrocarbons, cyclohexane is preferred since it selectively brings the salt partitioned in the organic solvent back into the aqueous phase, and since moreover, the impurities are left behind in the mixture of solvents. Most importantly, the solvent combination used helps maintain the diastereomeric ratio of (R/R+S) in the methanesulfonate salt between 0.5 to 0.6.

The aqueous layer containing the methanesulfonate salt (II¹) can be taken directly for the next neutralization step or can optionally be treated with carbon and filtered and then taken for the next neutralization step

In the neutralization step, the solution of the salt (II¹) in water is treated with a base to give pure cefpodoxime proxetil (I), which separates out from the medium and can be isolated by filtration.

The neutralization can be carried out with an inorganic base as well as an organic base. Inorganic bases are preferred as impurity formation associated with neutralization is higher when an organic base is used.

The inorganic bases that can be used include sodium hydroxide, sodium carbonate, sodium bicarbonate and potassium bicarbonate. Mild bases are preferred and among them, the alkali metal bicarbonates, like sodium hydrogen carbonate and potassium bicarbonate are preferred.

The molar range of sodium bicarbonate used for neutralization is in the range of 1.0 to 1.5, but preferably between 1.1 to 1.2 mole equivalent.

The base namely sodium bicarbonate is added as a solution in water, the concentration of the solution ranging from 4 to 7% but preferably between 5 to 6% solution.

The pH of the reaction mass after neutralization should be 7.0, as higher pH leads to degradation of the compound viz. cefpodoxime proxetil.

The time required for neutralization of the salt (II¹), varies between 15 to 45 minutes, but preferably 30 minutes.

The temperature range for neutralization of the salt is between 15 to 35° C., but preferably between 25 to 30° C.

The compound, viz. pure cefpodoxime proxetil, that separates out is filtered, and washed with water.

The diasteremeric ratio of the R and S isomer present in the pure cefpodoxime proxetil is in the range of 0.50 to 0.60 which conforms to the specified range of 0.5 to 0.6 mentioned in Pharmacopoeial Forum Vol. 28 (1), pp 44-52, (2002).

The pure cefpodoxime proxetil thus formed, has reduced level of impurities and conforms to pharmacopoeial specifications.

Optionally, the pure cefpodoxime proxetil as obtained above can be dissolved in a water-miscible organic solvent, followed by optional treatment of the solution with activated charcoal, followed by filtration through a filter aid to remove charcoal and suspended particles and addition of water to the filtrate to precipitate out cefpodoxime proxetil (I) free of impurities and having a diastereomeric ratio of (R/R+S) between 0.5 to 0.6, which can be isolated by filtration.

Examples of water-miscible organic solvents include lower alcohols such as methanol, ethanol and isopropanol; lower alkyl ketones such as acetone, lower alkyl glycols ethers such as methyl glycol; dipolar aprotic solvents such as N,N-dimethylacetamide, dimethyl sulfoxide and cyclic ethers such as tetrahydrofurran, dioxane etc. However, lower alcohols are preferred and specially methanol is preferred.

In a specific embodiment of the invention, impure cefpodoxime proxetil dissolved in ethyl acetate, is agitated at 25 to 30° C. A solution of methanesulphonic acid in water is added at the same temperature and stirred at the same temperature for 15 minutes. Cyclohexane is added to the mixture and stirred for 15 minutes. The aqueous layer is separated and stirred again with a mixture of ethyl acetate and cyclohexane. The aqueous layer is separated and optionally treated with activated carbon and filtered. The filtrate is stirred at 25 to 30° C. and a 6% solution of sodium bicarbonate in water is added to the mixture at 25 to 30° C. in 30 minutes to separate out pure cefpodoxime proxetil, which is isolated by filtration, conforming to pharmacopoeial specifications.

The invention can be further illustrated by the following examples, which, however, should not be construed as limiting the scope of the invention.

### EXAMPLE-1

Preparation of Cefpodoxime Proxetil (I):

Cefpodoxime acid (VI; 50 gms; 0.117 moles) was added to dimethyl acetamide (350 ml) and stirred to get a clear mixture. The mixture was cooled to −6 to −10° C. to which was added 1,8-diazabicyclo[5,4,0]undec-7-ene (DBU) (17.4 gms; 0.114 moles), followed by slow addition of 1-iodoethyl isopropyl carbonate (30.18 gms; 0.117 moles) over a period of 10 to 15 minutes. The reaction mixture was agitated for a period of 20 to 30 minutes at the same temperature. The reaction mixture was quenched by addition of 13% hydrochloric acid. The reaction mixture was further diluted with water (400 ml) and extracted with ethyl acetate (500 ml). The separated aqueous layer was re-extracted with ethyl acetate (500 ml). The organic layers were combined and stirred with 2% sodium carbonate solution (500 ml) at 0 to 5° C. for 30 minutes. The organic layer was washed with 5% sodium thiosulphate solution (500 ml). The organic layer was treated with charcoal (7.5 gms) at 25° C. for 60 minutes and filtered through celite bed. The filtrate was concentrated at reduced pressure and at a temperature, below 35° C. to give the title compound, having a isomer ratio of (R/R+S)=0.51

### EXAMPLE-2

Preparation of Cefpodoxime Proxetil (I):

A solution of cefpodoxime acid 25 g (0.058 moles) in dimethyl acetamido (175 ml) was cooled to −10° C. and 1,8 diazobicyclo[5,4,0]undec-7-ene 8.62 g (0.0567 moles) was added dropwise at −10 to −15° C. To this was added 1-iodoethyl isopropyl carbonate 14.5 g (0.0562 moles) dropwise at −10 to −6° C. over 10 minutes. The reaction mixture was stirred for 30 minutes at −10 to −6° C. and quenched by adding hydrochloric acid (5 ml) in de-mineralized water (45 ml) at −10° C.

The resulting reaction mire was poured into a solution of sodium bicarbonate (7 g) in de-mineralized water (525 ml) and cyclohexane (100 ml) at 0 to 10 (C. A white solid precipitated out which was stirred for 30 minutes and filtered, washed with de-mineralised water (100 ml×3 washes). The resulting slurry was given a wash by cyclohexane (100 ml) and the solid was dried at 40° C. under vacuum to obtain crude cefpodoxime proxetil 25 g (0.0448 moles) in 77% yield having a isomer ratio of (R/R+S)=0.52

### EXAMPLE-3

Preparation of Cefpodoxime Proxetil (I):

A solution of cefpodoxime acid (50 g, 0.0117 moles) in 350 ml of dimethylacetamide was cooled to −10° C. and 1,8 diazabicyclo[5,4,0]undec-7-ene (17.5 g, 0.0115 moles) was added dropwise at −10 to −15° C. To this was added 1-iodoethyl isopropyl carbonate 28.9 g (0.0112 moles) dropwise at −10 to −6° C. over 10-15 minutes. The reaction mixture was stirred for 30 minutes at −10 to −6° C. and quenched by adding hydrochloric acid (10 ml) in de-mineralized water (90 ml) at −10° C.

To the resulting reaction mixture 500 ml of ethyl acetate and a solution of sodium dithionate (3 g dissolved in 400 ml of de mineralizes water) was added. The organic layer was separated after 10 minutes stirring. The aqueous layer was extracted with ethyl acetate (500 ml). The combined organic layer was stirred with 2% aqueous sodium bicarbonate (500 ml) solution at 0-5° C. for 30 minutes. The organic layer was separated and washed with 5% aqueous sodium thiosulfate solution (500 ml). The organic layer was treated with activated carbon and filtered. The filtrate was concentrated up to 200 ml volume. The concentrated organic layer was added dropwise to 1000 ml of cyclohexane over 1 hour of time and then stirred for 30 minutes at room temperature. The solution was filtered and washed with cyclohexane (250 ml) and dried under vacuum at 40° C. to afford cefpodoxime proxetil (52 g) in 79.7% yield having a isomer ratio of (R/R+S)=0.53

### EXAMPLE-4

Purification of Crude Cefpodoxime Proxetil:

Cefpodoxime proxetil (5 g; 0.00897 moles), as obtained in Example-1, Example-2 or Example-3 was dissolved in ethyl acetate (12.5 ml) and stirred at 25-30° C. Methanesulphonic acid (1.5 g; 0.00156 moles) dissolved in water (75 ml) was added to the mixture, at 25-30° C. The mixture was stirred for 15 minutes. Cyclohexane (10 ml) was added to the mixture and stirred for 15 minutes. The two layers were separated, and to the aqueous layer was added ethyl acetate (12.5 ml) followed by cyclohexane (10 ml) and the mixture agitated for 15 minutes. The aqueous layer was separated and stirred with activated carbon (0.5 gms) for 45 minutes and filtered. The filtrate was stirred at 25-30° C., and a solution of 6% sodium bicarbonate in water was added to the mixture at the same temperature in 30 minutes to get pH: 7.0. The pure compound, separating out was filtered and washed with water (2×25 ml) and dried under vacuum to give pure cefpodoxime proxetil (3.53 g; yield: 65.3%), having an isomer ratio of (R/R+S)=0.52

### EXAMPLE-5

Purification of Crude Cefpodoxime Proxetil:

Crude cefpodoxime proxetil 25 g (0.0448 moles) obtained from example-1, example-2 or example-3 was dissolved in a solution of methane sulfonic acid 7.3 g (0.076 moles) and water (250 ml). To the solution was added ethyl acetate (62.5 ml) and stirred for 15 minutes at 27° C. Cyclohexane (50 ml) was added to the mixture at 27° C. and stirred for 15 minutes. The aqueous layer was separated and stirred with ethyl acetate (62.5 ml) for 15 minutes at 27° C. 50 ml of cyclohexane was added and the reaction mixture was stirred for 15 minutes at 27° C.

The aqueous layer was separated and stirred with 2.5 g of activated carbon for 30 min at 27° C. and filtered off. To the aqueous layer a solution of sodium bi carbonate (6% in water) was added at 25-27° C. and the pH was adjusted to 7. The reaction mixture was stirred for 30 minutes at 25-27° C. The precipitated solid was filtered off and washed by water (100 ml×3 times). The wet mass was dried at 40° C. under vacuum to yield 22 g (0.039 moles) of pure cefpodoxime proxetil having an isomer ratio of (R/R+S)=0.519

### EXAMPLE-6

One Pot Preparation and Purification of Cefpodoxime Proxetil (I):

Cefpodoxime acid 5 g (0.0117 moles) was dissolved in dimethylacetamide (35 ml). The solution was cooled to −10° C. 1.73 g (0.01138 moles) of 1,8 diazobicyclo[5,4,0]undec-7-ene was added dropwise over −10 to −15° C. 2.92 g of 1-iodoethyl isopropyl carbonate (0.01133 moles) was added at −10 to −6° C. within 10 minutes. The reaction mixture was stirred for 30 minutes and hydrochloric acid (1 ml HCl in 10 ml DMW) was added at −10 to +5° C. A solution of 0.3 g of sodium dithionate in 2 ml water was added at 0° C. This was followed by addition of ethyl acetate (50 ml) and water (38 ml) at 0° C. The aqueous and organic layers were separated. The organic layer was stirred with 2% NaHCO₃ in water (50 ml) for 30 minutes at 0-5° C. The organic layer was separated and stirred with 5% sodium thiosulfate (50 ml) for 10 minutes at 0 to 5° C. The organic layer was separated and treated with activated carbon (0.75 g) for 30 minutes and filtered.

The organic layer was concentrated up to 2.5 volumes under vacuum at 40° C. and cooled to 25° C. The solution of impure cefpodoxime proxetil in ethyl acetate was treated with a solution of methanesulfonic acid (1.5 g, 0.0156 moles) in water (50 ml) and stirred for 15 minutes at 25 to 27° C. 10 ml of cyclohexane was added to the reaction mixture and stirred for 15 minutes. The organic and aqueous layers were separated. The aqueous layer was treated with ethyl acetate (12 ml) for 15 minutes at 25 to 27° C. Cyclohexane (10 ml) was added to the reaction mixture and stirred for 15 minutes at 25 to 27° C. The aqueous layer was separated and neutralized by 6% NaHCO₃ solution til pH 7. The precipitated solid was filtered and washed with water (20 ml×3 times). The solid was filtered and dried under vacuum at 40° C. to give 3.4 g (0.006 moles) of cefpodoxime proxetil having an isomer ratio of (R/+S)=0.52.

1. A process for obtaining cefpodoxime proxetil of formula (I), of high purity conforming to pharmacopoeial specification comprising; 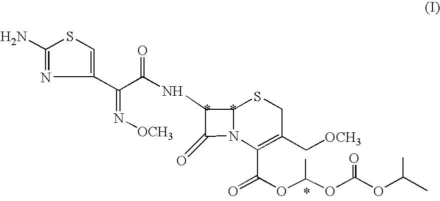
adding a solution of methanesulfonic acid in water to a solution of impure cefpodoxime proxetil of formula (I) in an organic solvent to form the corresponding cefpodoxime proxetil methanesulfonate of formula (II¹); 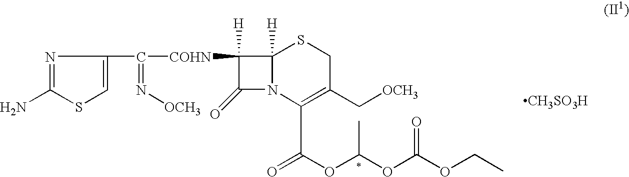
adding a co-solvent and separating of the aqueous phase comprising cefpodoxime proxetil methanesulfonate of formula (II¹) having a diastereomeric ratio of (R/R+S) between 0.5 to 0.6; neutralizing the methanesulfonate salt (II¹) with a base to give cefpodoxime proxetil (I) free of impurities and having a diastereomeric ratio of (R/R+S) between 0.5 to 0.6 or,
adding of impure cefpodoxime proxetil of formula (I) to a solution of methanesulfonate acid in water to form the corresponding solution of cefpodoxime proxetil methanesulfonate of formula (II¹) in water,

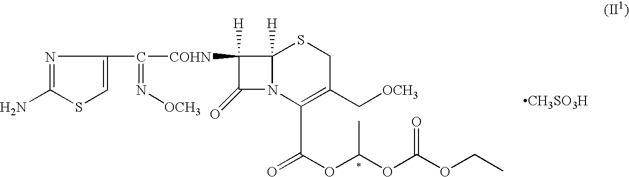

2. A process as claimed in claim 1, wherein said pure cefpodoxime proxetil is dissolved in a water-miscible organic solvent, followed by optional treatment of the solution with activated charcoal, followed by filtration through a filter aid to remove charcoal and suspended particles and addition of water to the filtrate to precipitate out cefpodoxime proxetil (I) free of impurities and having a diastereomeric ratio of (R/R+S) between 0.5 to 0.6, which can be isolated by filtration.

3. A process as claimed in claim 1, wherein said first organic solvent is a water-immiscible solvent.

4. A process as claimed in claim 3, wherein the water-immiscible organic solvent is selected from the group consisting of methyl acetate, ethyl acetate, butyl acetate, methyl ethyl ketone and methyl iso-butyl ketone.

5. A process as claimed in claim 1, wherein the co-solvent is selected from the group consisting of an aliphatic hydrocarbon, aromatic hydrocarbon and an ether.

6. A process as claimed in claim 5, wherein the aliphatic hydrocarbon is selected from the group consisting of hexane, heptane, cyclopentane and cyclohexane.

7. A process as claimed in claim 5, wherein the aromatic hydrocarbon is selected from toluene and xylene.

8. A process as claimed in claim 5, wherein the ether is diethyl ether or diisopropyl ether.

9. A process as claimed in claim L wherein said methanesulfonic acid is employed in a molar ratio of between 1.0 to 2.0 mole equivalent of cefpodoxime proxetil, preferably between 1.5 to 2.0 mole equivalent.

10. A process as claimed in claim 1 wherein the diastereomeric ratio of (R/R+S) the methanesulfonate salt of formula (II¹) obtained after separation of the organic and aqueous phases is between 0.5 to 0.6.

11. A process as claimed in claim 1, wherein the base is an inorganic base.

12. A process as claimed in claim 11, wherein the inorganic base is selected from the group consisting of sodium hydroxide, potassium hydroxide, sodium carbonate, potassium carbonate, sodium hydrogen carbonate and potassium hydrogen carbonate.

13. A process as claimed in claim 1, wherein the pH of the solution after neutralization with the base is 7.0.

14. A process as claimed in claim 1, wherein the pure cefpodoxime proxetil of formula (I) after neutralization with a base is isolated by filtration.

15. A process as claimed in claim 1, wherein the diastereomeric ratio of (R/R+S) pure cefpodoxime proxetil of formula (I) is between 0.5 to 0.6.

16. A process as claimed in claim 3, wherein the water-miscible organic solvent is selected from the group consisting of lower alcohols; lower alkyl ketones; lower alkyl glycols ethers; dipolar aprotic solvents and cyclic ethers.

17. (canceled)

18. A process as claimed in claim 9, wherein said methanesulfonic acid is employed in a molar ratio of between 1.0 to 2.0 mole equivalent of cefpodoxime proxetil, preferably between 1.5 to 2.0 mole equivalent.

19. The process according to claim 16, wherein the lower alcohol is selected from the group consisting of methanol, ethanol and isopropanol.

20. The process according to claim 16, wherein the lower alkyl ketone is acetone.

21. The process according to claim 16, wherein the lower alkyl glycol ether is methyl glycol.

22. The process according to claim 16, wherein the dipolar aprotic solvent is selected from the group consisting of N,N-dimethylacetamide and dimethyl sulfoxide.

23. The process according to claim 16, wherein the cyclic ether is selected from the group consisting of tetrahydrofuran and dioxane.
